(19) United States
(12) Patent Application Publication
(10) Pub. No.: US 2015/0333269 A1
(43) Pub. Date: Nov. 19, 2015

(54) SOLVENT OR SOLVENT COMPOSITION FOR MANUFACTURING ORGANIC TRANSISTOR

(71) Applicant: DAICEL CORPORATION, Osaka-shi, Osaka (JP)

(72) Inventors: Youji SUZUKI, Himeji-shi (JP); Takeshi YOKOO, Himeji-shi (JP); Yasuyuki AKAI, Himeji-shi (JP)

(73) Assignee: DAICEL CORPORATION, Osaka-shi, Osaka (JP)

(21) Appl. No.: 14/651,312

(22) PCT Filed: Dec. 2, 2013
(86) PCT No.: PCT/JP2013/082369

§ 371 Date: Jun. 11, 2015

(30) Foreign Application Priority Data
Dec. 12, 2012 (JP) 2012-271139

(51) Int. Cl. H01L 51/00; C07D 307/16

(52) U.S. Cl. CPC H01L 51/0065; C07D 307/16; H01L 51/0074; H01L 51/0508

(57) ABSTRACT

Provided is a solvent or solvent composition for organic transistor production. The solvent or solvent composition allows an organic semiconductor material to dissolve therein with high solubility and can form a highly crystalline organic transistor.

The solvent or solvent composition for organic transistor production according to the present invention is a solvent or solvent composition for dissolution of an organic semiconductor material and includes a solvent A represented by Formula (A). In Formula (A), R¹ is selected from C₁-C₄ alkyl, C₁-C₄ acyl, a C₅-C₆ cycloalkane ring, a C₅-C₆ cycloalkene ring, C₆-C₁₂ aryl, and a group including two or more of them bonded to each other. R², R³, R⁴, and R⁵ are, identically or differently in each occurrence, selected from hydrogen, C₁-C₄ alkyl, and C₁-C₄ acyl. R⁶ is selected from C₁-C₄ alkyl and C₁-C₄ acyl. R¹ and R³ may be linked to each other to form a ring with the adjacent oxygen atom and carbon atom; n represents 1 or 2; and m represents an integer from 0 to 2.

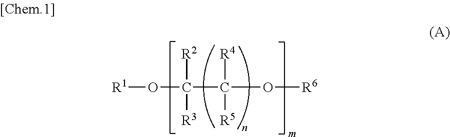

### TECHNICAL FIELD

The present invention relates to a solvent or solvent composition for organic transistor production, where the solvent or solvent composition allows an organic semiconductor material to dissolve therein with high solubility. The present invention also relates to a composition for organic transistor production, where the composition includes an organic semiconductor material and the solvent or solvent composition for organic transistor production. The present application claims priority to Japanese Patent Application No. 2012-271139 filed to Japan on Dec. 12, 2012, the entire contents of which are incorporated herein by reference.

### BACKGROUND ART

Transistors are widely used as important semiconductor electronic devices that constitute displays and computer devices. The transistors have employed polysilicons, amorphous silicon, and other inorganic substances as semiconductor materials. Disadvantageously, however, thin-film transistors using such inorganic substances require a vacuum process and/or a high-temperature process for their production and invite increased production cost. In addition, the production, as including a high-temperature process, has limitations on types of usable substrates and mainly employs glass substrates and similar substrates. Although having excellent heat resistance, the glass substrates are susceptible to impact, are hard to achieve weight reduction, have poor flexibility, and hardly give flexible transistors.

This has led to active investigations and development of organic electronic devices using organic semiconductor materials. Advantageously, the organic semiconductor materials can be easily formed into thin films by a simple procedure of a wet process such as printing or spin coating and can give organic transistors by a production process performed at a lower temperature as compared with conventional transistors using inorganic semiconductor materials. This enables the use of plastic substrates generally having relatively inferior heat resistance, achieves reduction in weight and cost of electronic devices such as displays, and is expected to be expanded variously, typically in uses utilizing flexibility of the plastic substrates.

The organic semiconductor materials are exemplified by low-molecular semiconductor materials such as dinaphtho[2,3-b:2′,3′-f]thieno[3,2-b]thiophene. The low-molecular semiconductor materials upon use are known to allow semiconductor devices to develop high performance (Non Patent Literature (NPL) 1). However, most of unsubstituted acene compounds typified by dinaphtho[2,3-b:2′,3′-f]thieno[3,2-b]thiophene have strong intermolecular interaction due to a n-conjugated system and thereby have poor solubility in a solvent (Patent Literature (PTL) 1). This impedes the preparation of a composition for organic transistor production containing the organic semiconductor material in a high concentration and causes an organic semiconductor formed by printing to include smaller crystal grains. Disadvantageously, the resulting organic semiconductor is not energized unless a high voltage is applied, and such high voltage upon application causes an insulating film to peel off.

As possible solutions to solve the problems, PTL 2 and PTL 3 describe techniques that employ, as organic semiconductor materials, acene compounds added with a leaving group so as to impart solubility to the materials. The techniques also employ halides (halogenated compounds) such as chloroform and dichlorobenzene as solvents. Unfortunately, however, most of the acene compounds added with a leaving group are more unstable to heating upon dissolution and have lower charge mobility as compared with acene compounds having no leaving group.

NPL 2 describes a technique that employs, as an organic semiconductor material, an acene compound added with an alkyl substituent so as to impart solubility, and, as a solvent, a halide that allows the organic semiconductor material to dissolve satisfactorily therein. Disadvantageously, however, the technique is inferior in operating safety because such halide has ecological toxicity concern.

PTL 4 describes a technique that employs a dispersion of an unsubstituted acene compound to form a thin film. However, it is difficult to prevent the aggregation of the unsubstituted acene compound contained in the dispersion and to maintain dispersibility of the unsubstituted acene compound. Unfortunately, therefore, the unsubstituted acene compound aggregates randomly to have inferior charge mobility.

### CITATION LIST

### Patent Literature

PTL 1: Japanese Unexamined Patent Application Publication (JP-A) No. 2009-302264

PTL 2: JP-A No. 2011-148743

PTL 3: JP-A No. 2012-041327

PTL 4: JP-A No. 2011-003852

### Non Patent Literature

NPL 1: J. Am. Chem. Soc., 2005, 127(14), pp. 614-618

NPL 2: J. Am. Chem. Soc., 2007, 129(14), pp. 15732-15733

### SUMMARY OF INVENTION

### Technical Problem

Accordingly, it is an object of the present invention to provide a solvent or solvent composition for organic transistor production, where the solvent or solvent composition allows an organic semiconductor material to dissolve therein with high solubility and can form a highly crystalline organic transistor.

It is another object of the present invention to provide a composition for organic transistor production, where the composition includes the solvent or solvent composition for organic transistor production.

### Solution to Problem

After intensive investigations to achieve the objects, the present inventors have found that a specific solvent or solvent composition, when used, allows an organic semiconductor material to dissolve therein with high solubility at a relatively low temperature and can form an organic transistor by a printing process even on a plastic substrate having lower heat resistance as compared with a glass substrate. The present inventors have also found that a composition for organic transistor production containing the solvent or solvent composition and an organic semiconductor material, when applied onto a substrate, allows the organic semiconductor material to undergo self-assembly to thereby crystallize. In addition, the present inventors have found that the solvent or solvent composition, when further containing a solvent generally used for electronic materials as needed, can have still better coatability and drying behavior. The present invention has been made based on these findings.

Specifically, the present invention provides, in an aspect, a solvent or solvent composition for organic transistor production. The solvent or solvent composition is for use in the dissolution of an organic semiconductor material and includes a solvent A represented by Formula (A):

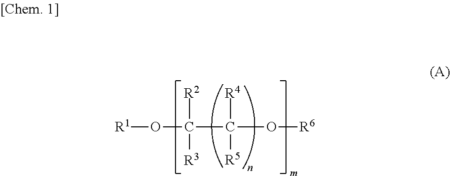

where R¹ is selected from C₁-C₄ alkyl, C₁-C₄ acyl, a C₅-C₆ cycloalkane ring, a C₅-C₆ cycloalkene ring, C₆-C₁₂ aryl, and a group including two or more of them bonded to each other; R², R³, R⁴, and R⁵ are, identically or differently in each occurrence, selected from hydrogen, C₁-C₄ alkyl, and C₁-C₄ acyl; R⁶ is selected from C₁-C₄ alkyl and C₁-C₄ acyl, where R¹ and R³ may be linked to each other to form a ring with adjacent oxygen atom and carbon atom; n represents 1 or 2; and m represents an integer from 0 to 2.

The solvent A preferably includes at least one selected from the group consisting of ethylene glycol dimethyl ether, diethylene glycol dimethyl ether, ethylene glycol monoethyl ether acetate, ethylene glycol monopropyl ether acetate, ethylene glycol monobutyl ether acetate, diethylene glycol monoethyl ether acetate, diethylene glycol monobutyl ether acetate, propylene glycol dimethyl ether, propylene glycol methyl ethyl ether, propylene glycol methyl propyl ether, propylene glycol methyl butyl ether, dipropylene glycol dimethyl ether, dipropylene glycol methyl ethyl ether, dipropylene glycol methyl propyl ether, dipropylene glycol methyl butyl ether, propylene glycol monomethyl ether acetate, propylene glycol monoethyl ether acetate, dipropylene glycol monomethyl ether acetate, dipropylene glycol monoethyl ether acetate, 3-methoxybutanol acetate, tetrahydrofurfuryl acetate, and cyclohexanol acetate.

The organic semiconductor material preferably includes at least one compound selected from the group consisting of compounds (1) and compounds (2). The compounds (1) are each represented by Formula (1):

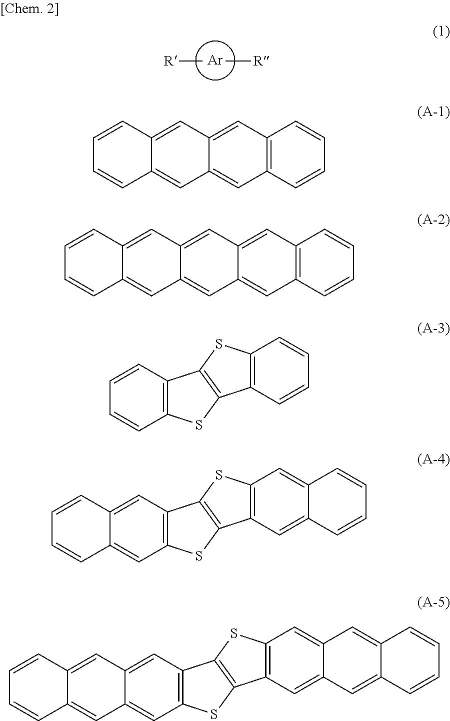

where Ar represents a group corresponding to a cyclic compound, except for removing two hydrogen atoms from the cyclic compound, where the cyclic compound is a compound represented by any one of Formulae (A-1) to (A-5); and R′ and R″ are each, identically or differently, selected from hydrogen, optionally substituted C₁-C₁₈ alkyl, optionally substituted phenyl, optionally substituted naphthyl, and optionally substituted thiophenyl. The compounds (2) each include at least one constitutional repeating unit represented by any one of Formulae (2-a) to (2-d):

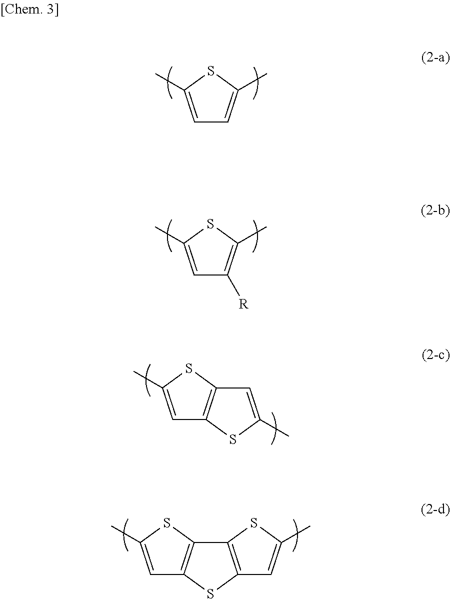

where R is selected from optionally substituted C₁-C₂₄ alkyl, optionally substituted phenyl, optionally substituted naphthyl, and optionally substituted thiophenyl.

The present invention provides, in another aspect, a composition for organic transistor production. The composition includes an organic semiconductor material and the solvent or solvent composition for organic transistor production.

The organic semiconductor material preferably includes at least one compound selected from the group consisting of compounds (1) and compounds (2). The compounds (1) are each represented by Formula (1):

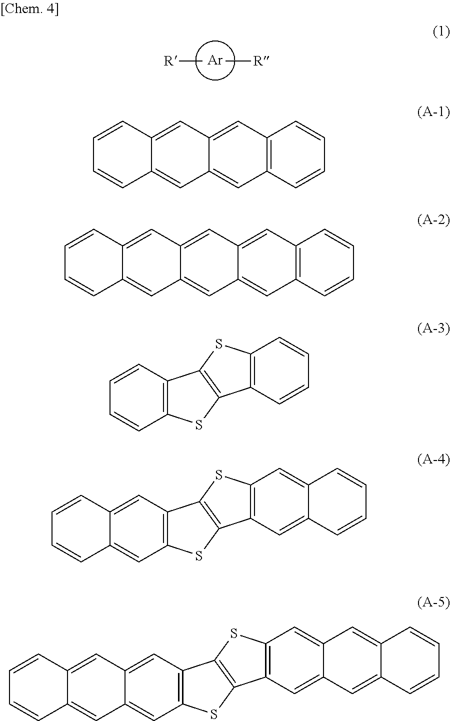

where Ar represents a group corresponding to a cyclic compound, except for removing two hydrogen atoms from the cyclic compound, where the cyclic compound is a compound represented by any one of Formulae (A-1) to (A-5); and R′ and R″ are each, identically or differently, selected from hydrogen, optionally substituted C₁-C₁₈ alkyl, optionally substituted phenyl, optionally substituted naphthyl, and optionally substituted thiophenyl. The compounds (2) each include at least one constitutional repeating unit represented by any one of Formulae (2-a) to (2-d):

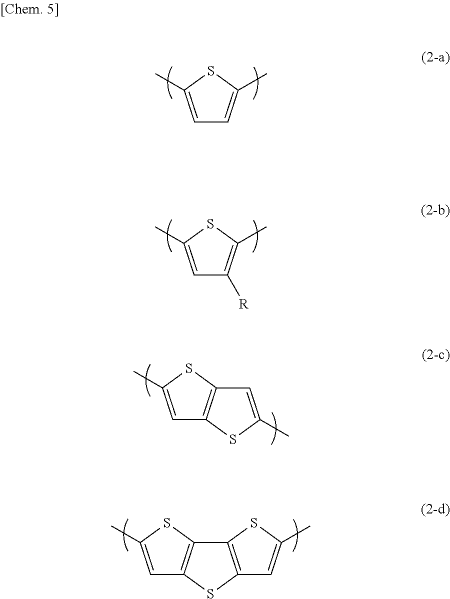

where R is selected from optionally substituted C₁-C₂₄ alkyl, optionally substituted phenyl, optionally substituted naphthyl, and optionally substituted thiophenyl.

Specifically, the present invention relates to followings.

(1) The present invention relates to a solvent or solvent composition for organic transistor production. The solvent or solvent composition is used for the dissolution of an organic semiconductor material and includes the solvent A represented by Formula (A).

(2) In the solvent or solvent composition for organic transistor production according to (1), the solvent A may include at least one selected from the group consisting of ethylene glycol dimethyl ether, diethylene glycol dimethyl ether, ethylene glycol monoethyl ether acetate, ethylene glycol monopropyl ether acetate, ethylene glycol monobutyl ether acetate, diethylene glycol monoethyl ether acetate, diethylene glycol monobutyl ether acetate, propylene glycol dimethyl ether, propylene glycol methyl ethyl ether, propylene glycol methyl propyl ether, propylene glycol methyl butyl ether, dipropylene glycol dimethyl ether, dipropylene glycol methyl ethyl ether, dipropylene glycol methyl propyl ether, dipropylene glycol methyl butyl ether, propylene glycol monomethyl ether acetate, propylene glycol monoethyl ether acetate, dipropylene glycol monomethyl ether acetate, dipropylene glycol monoethyl ether acetate, 3-methoxybutanol acetate, tetrahydrofurfuryl acetate, and cyclohexanol acetate.

(3) In the solvent or solvent composition for organic transistor production according to one of (1) and (2), the organic semiconductor material may include at least one compound selected from the group consisting of the compounds (1) represented by Formula (1) and the compounds (2) including at least one constitutional repeating unit represented by any one of Formulae (2-a) to (2-d).

(4) The present invention also relates to a composition for organic transistor production. The composition includes an organic semiconductor material and the solvent for organic transistor production according to one of (1) and (2).

(5) In the composition for organic transistor production according to (4), the organic semiconductor material may include at least one compound selected from the group consisting of the compounds (1) represented by Formula (1) and the compounds (2) including at least one constitutional repeating unit represented by any one of Formulae (2-a) to (2-d).

### Advantageous Effects of Invention

The solvent or solvent composition for organic transistor production according to the present invention allows an organic semiconductor material to dissolve therein with high solubility even at a relatively low temperature. The solvent or solvent composition therefore enables direct formation of an organic transistor even on, for example, a plastic substrate and enables the formation of displays and computer devices that are impact-resistant, lightweight, and flexible. This is because the plastic substrate is impact-resistant, lightweight, and flexible, although having relatively low heat resistance, as compared with a glass substrate. The solvent or solvent composition also enables easy production of organic transistors by a simple procedure of a wet process such as printing or spin coating and can provide significant cost reduction.

The composition for organic transistor production according to the present invention, when applied onto a substrate, allows the organic semiconductor material to undergo self-assembly to thereby crystallize to thereby give a highly crystalline organic transistor.

### DESCRIPTION OF EMBODIMENTS

Solvent or Solvent Composition for Organic Transistor Production

The solvent or solvent composition for organic transistor production according to the present invention is a solvent or solvent composition used for the dissolution of an organic semiconductor material and includes the solvent A represented by Formula (A).

Solvent A

The solvent A for use in the present invention is represented by Formula (A). In Formula (A), R¹ is selected from C₁-C₄ alkyl, C₁-C₄ acyl, a C₅-C₆ cycloalkane ring, a C₅-C₆ cycloalkene ring, C₆-C₁₂ aryl, and a group including two or more of them bonded to each other. R², R³, R⁴, and R⁵ are, identically or differently in each occurrence, selected from hydrogen, C₁-C₄ alkyl, and C₁-C₄ acyl. R⁶ is selected from C₁-C₄ alkyl and C₁-C₄ acyl. R¹ and R³ may be linked to each other to form a ring with the adjacent oxygen atom and carbon atom. The number n is 1 or 2, and m is an integer from 0 to 2.

The C₁-C₄ alkyl as R¹ to R⁶ refers to alkyl containing 1 to 4 carbon atoms and is exemplified by methyl, ethyl, propyl, and butyl.

The C₁-C₄ acyl as R¹ to R⁶ refers to acyl containing 1 to 4 carbon atoms and is exemplified by acetyl, propionyl, and butyryl.

As R¹, the C₅-C₆ cycloalkane ring refers to a cycloalkane ring having 5 or 6 carbon atoms and is exemplified by cyclopentane and cyclohexane rings; and the C₅-C₆ cycloalkene ring refers to a cycloalkene ring having 5 or 6 carbon atoms and is exemplified by cyclopentene and cyclohexene rings.

The C₆-C₁₂ aryl as R¹ refers to aryl containing 6 to 12 carbon atoms and is exemplified by phenyl and naphthyl.

In an embodiment, R¹ and R³ are linked to each other to form a ring with the adjacent oxygen atom and carbon atom. The ring is exemplified by tetrahydrofuran ring and other heterocyclic compounds including 5 to 7 members and containing an oxygen atom as a heteroatom.

The solvent A for use in the present invention is exemplified by ethylene glycol dimethyl ether, ethylene glycol diethyl ether, ethylene glycol dipropyl ether, ethylene glycol dibutyl ether, ethylene glycol methyl ethyl ether, ethylene glycol methyl propyl ether, ethylene glycol methyl butyl ether, ethylene glycol ethyl propyl ether, ethylene glycol ethyl butyl ether, ethylene glycol propyl butyl ether, diethylene glycol dimethyl ether, diethylene glycol diethyl ether, diethylene glycol dipropyl ether, diethylene glycol dibutyl ether, diethylene glycol methyl ethyl ether, diethylene glycol methyl propyl ether, diethylene glycol methyl butyl ether, diethylene glycol ethyl propyl ether, diethylene glycol ethyl butyl ether, diethylene glycol propyl butyl ether, ethylene glycol monomethyl ether acetate, ethylene glycol monoethyl ether acetate, ethylene glycol monopropyl ether acetate, ethylene glycol monobutyl ether acetate, diethylene glycol monomethyl ether acetate, diethylene glycol monoethyl ether acetate, diethylene glycol monopropyl ether acetate, diethylene glycol monobutyl ether acetate, propylene glycol dimethyl ether, propylene glycol diethyl ether, propylene glycol dipropyl ether, propylene glycol dibutyl ether, propylene glycol methyl ethyl ether, propylene glycol methyl propyl ether, propylene glycol methyl butyl ether, propylene glycol ethyl propyl ether, propylene glycol ethyl butyl ether, propylene glycol propyl butyl ether, dipropylene glycol dimethyl ether, dipropylene glycol diethyl ether, dipropylene glycol dipropyl ether, dipropylene glycol dibutyl ether, dipropylene glycol methyl ethyl ether, dipropylene glycol methyl propyl ether, dipropylene glycol methyl butyl ether, dipropylene glycol ethyl propyl ether, dipropylene glycol ethyl butyl ether, dipropylene glycol propyl butyl ether, propylene glycol monomethyl ether acetate, propylene glycol monoethyl ether acetate, propylene glycol monopropyl ether acetate, propylene glycol monobutyl ether acetate, dipropylene glycol monomethyl ether acetate, dipropylene glycol monoethyl ether acetate, dipropylene glycol monopropyl ether acetate, dipropylene glycol monobutyl ether acetate, 3-methoxybutanol acetate, tetrahydrofurfuryl acetate, methyl acetate, ethyl acetate, n-propyl acetate, isopropyl acetate, butyl acetate, cyclohexanol acetate, ethylene glycol diacetate, diethylene glycol diacetate, propylene glycol diacetate, dipropylene glycol diacetate, and 1,3-butylene glycol diacetate. The solvent A may include each of them alone or in combination.

Among them, preferred herein is at least one compound selected from the group consisting of ethylene glycol dimethyl ether, diethylene glycol dimethyl ether, ethylene glycol monoethyl ether acetate, ethylene glycol monopropyl ether acetate, ethylene glycol monobutyl ether acetate, diethylene glycol monoethyl ether acetate, diethylene glycol monobutyl ether acetate, propylene glycol dimethyl ether, propylene glycol methyl ethyl ether, propylene glycol methyl propyl ether, propylene glycol methyl butyl ether, dipropylene glycol dimethyl ether, dipropylene glycol methyl ethyl ether, dipropylene glycol methyl propyl ether, dipropylene glycol methyl butyl ether, propylene glycol monomethyl ether acetate, propylene glycol monoethyl ether acetate, dipropylene glycol monomethyl ether acetate, dipropylene glycol monoethyl ether acetate, 3-methoxybutanol acetate, tetrahydrofurfuryl acetate, and cyclohexanol acetate. These are preferred because of allowing an organic semiconductor material to satisfactorily dissolve therein.

The solvent or solvent composition for organic transistor production may contain the solvent A in a content of preferably 50 percent by weight or more (e.g., 50 to 100 percent by weight) and particularly preferably 70 percent by weight or more (e.g., 70 to 100 percent by weight) based on the total amount (100 percent by weight) of the solvent or solvent composition. When the solvent A includes two or more different solvents, the “content” herein refers the total content of the two or more solvents. The solvent or solvent composition, if containing the solvent A in a content less than the range, may readily cause an organic semiconductor material to dissolve therein with insufficient solubility.

Solvent B The solvent or solvent composition for organic transistor production according to the present invention may further include a solvent B in combination with the solvent A. The “solvent B” refers to a solvent generally used for electronic materials and is compatible or miscible with the solvent A.

The solvent B is exemplified by (mono-, di-, or tri-) alkylene glycol monoalkyl ethers, C₃-C₆ alcohols, C₃-C₆ alkanediols, C₃-C₆-alkanediol monoalkyl ethers, C₃-C₆-alkanediol alkyl ether acetates, C₄-C₆-alkanediol diacetates, glycerol triacetate, hydroxycarboxylic acid esters, hydroxycarboxylic acid diesters, alkoxycarboxylic acid esters, cyclic ketones, lactones, cyclic ethers, amides, pyridines, aromatic hydrocarbons, aromatic acetates, aromatic ethers, and amines. The solvent B may include each of them alone or in combination.

The (mono-, di-, or tri-)alkylene glycol monoalkyl ethers are exemplified by ethylene glycol monomethyl ether, ethylene glycol monoethyl ether, ethylene glycol n-propyl ether, ethylene glycol n-butyl ether, diethylene glycol monomethyl ether, diethylene glycol monoethyl ether, diethylene glycol n-propyl ether, diethylene glycol n-butyl ether, propylene glycol monomethyl ether, propylene glycol monoethyl ether, propylene glycol n-propyl ether, propylene glycol n-butyl ether, dipropylene glycol monomethyl ether, dipropylene glycol monoethyl ether, dipropylene glycol n-propyl ether, dipropylene glycol n-butyl ether, tripropylene glycol monomethyl ether, and tripropylene glycol n-butyl ether.

The C₃-C₆ alcohols are exemplified by n-propyl alcohol, isopropyl alcohol, n-butyl alcohol, sec-butyl alcohol, tert-butyl alcohol, n-pentyl alcohol, n-hexyl alcohol, and 2-hexyl alcohol.

The C₃-C₆ alkanediols are exemplified by 1,3-butylene glycol, 1,4-butanediol, and 1,6-hexanediol.

The C₃-C₆-alkanediol monoalkyl ethers are exemplified by 3-methoxybutanol.

The C₃-C₆-alkanediol alkyl ether acetates are exemplified by 3-methoxybutyl acetate.

The C₄-C₆-alkanediol diacetates are exemplified by 1,4-butanediol diacetate and 1,6-hexanediol diacetate.

The hydroxycarboxylic acid esters are exemplified by methyl lactate and ethyl lactate.

The hydroxycarboxylic acid diesters are exemplified by methyl lactate acetate and ethyl lactate acetate.

The alkoxycarboxylic acid ester are exemplified by methoxymethyl propionate, and ethoxyethyl propionate.

The cyclic ketones are exemplified by cyclopentanone, cyclohexanone, and 4-ketoisophorone.

The lactones are exemplified by β-butyrolactone, γ-butyrolactone, ε-caprolactone, δ-valerolactone, γ-valerolactone, and α-acetyl-γ-butyrolactone.

The cyclic ethers are exemplified by tetrahydrofuran and tetrahydrofurfuryl alcohol.

The amides are exemplified by dimethylformamide.

The pyridines are exemplified by pyridine and methylpyridine.

The aromatic hydrocarbon are exemplified by toluene and tetralin.

The aromatic acetates are exemplified by phenyl acetate.

The aromatic ethers are exemplified by anisole.

The amines are exemplified by diethylamine and triethylamine.

In an embodiment of the present invention, the solvent A and the solvent B are used in combination. This gives a composition for organic transistor production, where the composition contains an organic semiconductor material in a high concentration and has excellent properties such as coatability and drying behavior.

For better coatability, it is effective to use one or more solvents selected from the group consisting of the (mono-, di-, or tri-)alkylene glycol monoalkyl ethers and alkoxycarboxylic acid esters in combination with the solvent A.

For better drying behavior, it is effective to use one or more solvents selected from the group consisting of cyclic ketones, cyclic ethers, aromatic hydrocarbons, aromatic acetates, and aromatic ethers in combination with the solvent A.

In the combination use, the ratio (ratio of amount in weight) of the solvent A to the solvent B is typically from 95:5 to 50:50, and preferably from 95:5 to 70:30. The solvent or solvent composition, if containing the solvent B in a large proportion with respect to the solvent A, may readily cause an organic semiconductor material to dissolve therein with insufficient solubility. When the solvent A includes two or more different solvents, the “amount” refers to the total amount of the two or more solvents. The same is applied to the solvent B.

The solvent or solvent composition for organic transistor production according to the present invention, as containing the solvent A, allows an organic semiconductor material to dissolve therein with high solubility. For example, the compound represented by Formula (1) may have a solubility in the solvent or solvent composition for organic transistor production at 100° C. of typically 0.05 part by weight or more, preferably 0.06 part by weight or more, and particularly preferably 0.07 part by weight or more per 100 parts by weight of the solvent or solvent composition for organic transistor production. The upper limit of the solubility is typically 0.5 part by weight, preferably 0.4 part by weight, and particularly preferably 0.3 part by weight.

Organic Semiconductor Material

The solvent or solvent composition for organic transistor production according to the present invention is a solvent or solvent composition for the dissolution of an organic semiconductor material. The organic semiconductor material is not limited, but preferably includes at least one compound selected from the group consisting of the compounds (1) represented by Formula (1) and the compounds (2) including at least one constitutional repeating unit represented by any one of Formulae (2-a) to (2-d). The organic semiconductor material may include each of them alone or in combination.

In Formula (1), Ar represents a group corresponding to a cyclic compound, except for removing two hydrogen atoms from the cyclic compound, where the cyclic compound is a compound represented by any one of Formulae (A-1) to (A-5). R′ and R″ are each, identically or differently, selected from hydrogen, optionally substituted C₁-C₁₈ alkyl, optionally substituted phenyl, optionally substituted naphthyl, and optionally substituted thiophenyl.

The C₁-C₁₈ alkyl refers to alkyl containing 1 to 18 carbon atoms and is exemplified by straight or branched chain alkyl such as methyl, ethyl, propyl, butyl, pentyl, hexyl, octyl, ethylhexyl, decyl, dodecyl, myristyl, hexyldecyl, and octyldecyl. The C₁-C₁₈ alkyl may bear one or more substituents. The substituents are exemplified by C₆-C₁₀ aryl such as phenyl and naphthyl. The phenyl, naphthyl, and thiophenyl may each bear one or more substituents. The substituents are exemplified by C₁-C₁₂ straight or branched chain alkyl such as methyl, ethyl, n-propyl, isopropyl, n-butyl, sec-butyl, and t-butyl.

Of the compounds (1) represented by Formula (1), preferred is the compound represented by Formula (1-1) so as to give an organic transistor including large crystal grains. Formula (1-1) is expressed as follows:

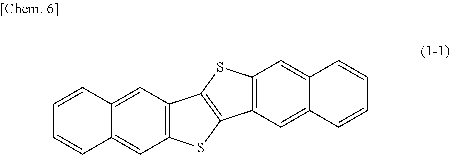

In Formula (2-b), R is selected from optionally substituted C₁-C₂₄ alkyl, optionally substituted phenyl, optionally substituted naphthyl, and optionally substituted thiophenyl.

The C₁-C₂₄ alkyl refers to alkyl containing 1 to 24 carbon atoms and is exemplified by straight or branched chain alkyl such as methyl, ethyl, propyl, butyl, pentyl, hexyl, octyl, ethylhexyl, decyl, dodecyl, myristyl, hexyldecyl, octyldecyl, icosyl, and tetracosyl.

The C₁-C₂₄ alkyl, phenyl, naphthyl, and thiophenyl as R may each bear one or more substituents. The substituents are exemplified as in R′ and R″.

The compounds (2) may each have a number of constitutional repeating units (degree of polymerization) of typically preferably from about 2 to about 5000. When the compounds (2) each include two or more different constitutional repeating units, the individual constitutional repeating units may be bonded to each other randomly or regularly.

Of the compounds (2), preferred are compounds including at least one constitutional repeating unit represented by any one of Formulae (2-1) to (2-3) so as to give an organic transistor containing large crystal grains. In the formulae, k, 1, and m each independently represent a number of the constitutional repeating unit indicated in the brackets and are an integer from 2 to 5000. Formulae (2-1) to (2-3) are expressed as follows:

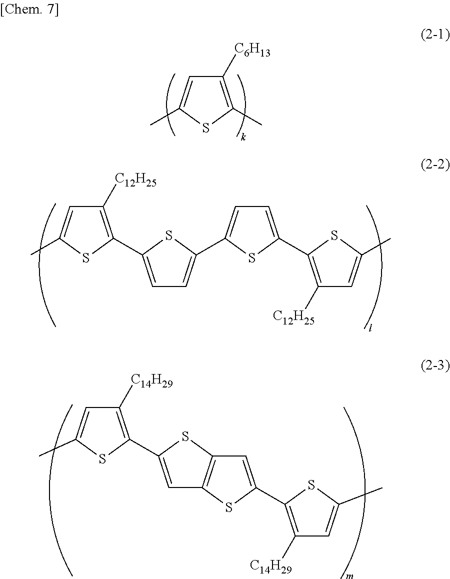

Composition for Organic Transistor Production

The composition for organic transistor production according to the present invention includes the organic semiconductor material and the solvent or solvent composition for organic transistor production.

The composition for organic transistor production according to the present invention may be prepared typically by mixing the organic semiconductor material with the solvent or solvent composition for organic transistor production and heating the mixture at a temperature of about 70° C. to about 150° C. in a nitrogen atmosphere for about 0.1 to about 10 hours under light-blocking conditions.

The composition for organic transistor production according to the present invention may contain the organic semiconductor material (in particular, the compound represented by Formula (1)) in a content of typically 0.05 percent by weight or more, preferably 0.06 percent by weight or more, and particularly preferably 0.07 percent by weight or more, based on the total amount (100 percent by weight) of the composition. When the organic semiconductor material includes two or more different materials, the term “content” refers to the total content of the two or different materials. The upper limit of the content is typically 0.5 percent by weight, preferably 0.4 percent by weight, and particularly preferably 0.3 percent by weight.

The composition for organic transistor production according to the present invention may contain the solvent or solvent composition for organic transistor production in a content of typically 99.99 percent by weight or less based on the total amount (100 percent by weight) of the composition. When the solvent or solvent composition includes two or more different solvents or solvent compositions, the term “content” refers to the total content of the two or more different solvents or solvent compositions. The lower limit of the content is typically 92.00 percent by weight, preferably 95.00 percent by weight, and particularly preferably 95.50 percent by weight, and the upper limit of the content is preferably 99.98 percent by weight, and particularly preferably 99.96 percent by weight.

Specifically, the composition for organic transistor production according to the present invention may contain the solvent or solvent composition for organic transistor production in an amount typically preferably 200 times (by weight) or more, more preferably 250 times (by weight) or more, and particularly preferably 333 times (by weight) or more the amount of the organic semiconductor material (in particular the amount of the compound represented by Formula (1)) contained in the composition for organic transistor production according to the present invention. This is preferred so as to accelerate the self-assembly and resulting crystallization of the organic semiconductor material. When the solvent or solvent composition includes two or more different solvents or solvent compositions, the “amount of the solvent or solvent composition” refers to the total amount of the two or more different solvents or solvent compositions. When the organic semiconductor material includes two or more different materials, the “amount of the organic semiconductor material” refers to the total amount of the two or more different materials. The upper limit of the amount of the solvent or solvent composition is typically 2000 times (by weight), preferably 1667 times (by weight), and particularly preferably 1429 times (by weight) the amount of the organic semiconductor material.

The composition for organic transistor production according to the present invention may further include, as needed and as appropriate, one or more other components that may be contained in regular compositions for organic transistor production, in addition to the organic semiconductor material and the solvent or solvent composition for organic transistor production. The other components are exemplified by raw materials for resins such as epoxy resins, acrylic resins, cellulosic resins, and butyral resins.

The composition for organic transistor production according to the present invention can contain the organic semiconductor material as dissolved in a high concentration even at a relatively low temperature. The composition can therefore form an organic transistor even directly on a plastic substrate to form displays and computer devices that are impact-resistant, lightweight, and flexible. This is because, although having lower heat resistance, the plastic substrate is more impact-resistant, has a lighter weight, and is more flexible as compared with a glass substrate. The composition for organic transistor production according to the present invention includes the solvent or solvent composition for organic transistor production according to the present invention and, when applied onto a substrate, allows the organic semiconductor material to undergo self-assembly and resulting crystallization and gives a highly crystalline organic transistor. In addition, the composition can easily form an organic transistor by a simple procedure of a wet process such as printing or spin coating and achieves significant cost reduction.

### EXAMPLES

The present invention will be illustrated in further detail with reference to several examples below. It should be noted, however, that the examples are by no means intended to limit the scope of the present invention.

### Example 1

An organic semiconductor material and a solvent for organic transistor production used herein were respectively dinaphtho[2,3-b:2′,3′-f]thieno[3,2-b]thiophene (DNTT; the compound represented by Formula (1-1); supplied by Wako Pure Chemical Industries, Ltd.) and tetrahydrofurfuryl acetate (THFFA; supplied by Daicel Corporation).

The organic semiconductor material was dispersed to a concentration of 0.08 to 0.15 percent by weight in the solvent for organic transistor production at an ambient temperature of 20° C. The dispersion was heated at 100° C. in a nitrogen atmosphere for about 6 hours under light-blocking conditions and yielded a composition for organic transistor production. The resulting composition for organic transistor production was examined on insoluble matter by visual observation, and the solubility of the organic semiconductor material was evaluated according to criteria as follows.

Criteria

Good (dissolved): No insoluble matter was observed; and

Poor (undissolved): Insoluble matter was observed.

### Examples 2 to 9 and Comparative Example 1

A composition for organic transistor production was prepared, and the solubility of an organic semiconductor material contained in the composition was evaluated by the procedure of Example 1, except for using a solvent for organic transistor production as given in Table 1.

TABLE 1

| | | DNTT concentration | | | |
|---|---|---|---|---|---|
| | Solvent for organic | (in weight percent) | | | |
| | transistor production | 0.08 | 0.09 | 0.10 | 0.15 |
| Example 1 | THFFA | Good | Good | Good | Good |
| Example 2 | EDGAC | Good | Good | Good | Good |
| Example 3 | DPMA | Good | Good | Good | Good |
| Example 4 | PMNP | Good | Poor | Poor | Poor |
| Example 5 | PMNB | Good | Poor | Poor | Poor |
| Example 6 | DMM | Good | Poor | Poor | Poor |
| Example 7 | DPMNP | Good | Poor | Poor | Poor |
| Example 8 | DPMNB | Good | Poor | Poor | Poor |
| Example 9 | MBA | Good | Poor | Poor | Poor |
| Comparative | o-DCB | Poor | Poor | Poor | Poor |
| Example 1 | | | | | |

THFFA: Tetrahydrofurfuryl acetate (supplied by Daicel Corporation)
EDGAC: Diethylene glycol monoethyl ether acetate (supplied by Daicel Corporation)
DPMA: Dipropylene glycol monomethyl ether acetate (supplied by Daicel Corporation)
PMNP: Propylene glycol methyl n-propyl ether (supplied by Daicel Corporation)
PMNB: Propylene glycol methyl n-butyl ether (supplied by Daicel Corporation)
DMM: Dipropylene glycol dimethyl ether (supplied by Daicel Corporation)
DPMNP: Dipropylene glycol methyl n-propyl ether (supplied by Daicel Corporation)
DPMNB: Dipropylene glycol methyl n-butyl ether (supplied by Daicel Corporation)
MBA: 3-Methoxybutanol acetate (supplied by Daicel Corporation)
o-DCB: 1,2-Dichlorobenzene (supplied by Tokyo Chemical Industry Co., Ltd.)

### Example 10

An organic semiconductor material and a solvent for organic transistor production used herein were respectively poly(3-hexylthiophene-2,5-diyl) (regioregular) (P3HT; the compound represented by Formula (2-1)) and cyclohexanol acetate.

The organic semiconductor material was dispersed to a concentration of 0.50 percent by weight in the solvent for organic transistor production at an ambient temperature of 20° C. The dispersion was heated at 100° C. in a nitrogen atmosphere for about 6 hours under light-blocking conditions and yielded a composition for organic transistor production.

The resulting composition for organic transistor production included no insoluble matter as observed.

As is demonstrated by Examples 1 to 9, the solvents for organic transistor production according to the present invention allow the organic semiconductor material (the compound represented by Formula (1), in particular, dinaphtho[2,3-b:2′,3′-f]thieno[3,2-b]thiophene: DNTT) to dissolve therein with excellent solubility as compared with 1,2-dichlorobenzene (o-DCB) that has been conventionally used.

As demonstrated by Example 10, the solvent for organic transistor production according to the present invention allows even poly(3-hexylthiophene-2,5-diyl) (regioregular) (P3HT: the compound represented by Formula (2-1)) to dissolve therein with excellent solubility.

1,2-Dichlorobenzene (o-DCB) has toxicity and is hard to handle. In contrast, the solvents for organic transistor production according to the present invention can be handled easily and satisfactorily.

### INDUSTRIAL APPLICABILITY

The solvent or solvent composition for organic transistor production according to the present invention allows an organic semiconductor material to dissolve therein with high solubility even at a relatively low temperature. The solvent or solvent composition can therefore form an organic transistor even directly on, for example, a plastic substrate and can form displays and computer devices that are impact-resistant, lightweight, and flexible. This is because the plastic substrate, although having lower heat resistance, is more impact-resistant, has a lighter weight, and is more flexible as compared to a glass substrate. The solvent or solvent composition also enables easy production of an organic transistor by a simple procedure of a wet process such as printing or spin coating and achieves significant cost reduction.

The composition for organic transistor production according to the present invention, when applied onto a substrate, allows the organic semiconductor material to undergo self-assembly and resulting crystallization and gives a highly crystalline organic transistor.

1. A solvent or solvent composition for organic transistor production, for use in dissolution of an organic semiconductor material, the solvent or solvent composition comprising
a solvent A represented by Formula (A): 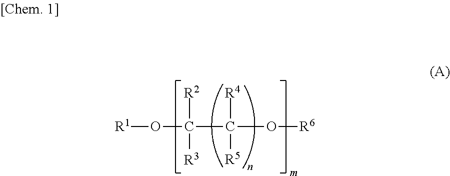
wherein R¹ is selected from C₁-C₄ alkyl, C₁-C₄ acyl, a C₅-C₆ cycloalkane ring, a C₅-C₆ cycloalkene ring, C₆-C₁₂ aryl, and a group comprising two or more of them bonded to each other; R², R³, R⁴, and R⁵ are, identically or differently in each occurrence, selected from hydrogen, C₁-C₄ alkyl, and C₁-C₄ acyl; R⁶ is selected from C₁-C₄ alkyl and C₁-C₄ acyl, where R¹ and R³ may be linked to each other to form a ring with adjacent oxygen atom and carbon atom; n represents 1 or 2; and m represents an integer from 0 to 2.

2. The solvent or solvent composition for organic transistor production according to claim 1,
wherein the solvent A comprises at least one selected from the group consisting of ethylene glycol dimethyl ether, diethylene glycol dimethyl ether, ethylene glycol monoethyl ether acetate, ethylene glycol monopropyl ether acetate, ethylene glycol monobutyl ether acetate, diethylene glycol monoethyl ether acetate, diethylene glycol monobutyl ether acetate, propylene glycol dimethyl ether, propylene glycol methyl ethyl ether, propylene glycol methyl propyl ether, propylene glycol methyl butyl ether, dipropylene glycol dimethyl ether, dipropylene glycol methyl ethyl ether, dipropylene glycol methyl propyl ether, dipropylene glycol methyl butyl ether, propylene glycol monomethyl ether acetate, propylene glycol monoethyl ether acetate, dipropylene glycol monomethyl ether acetate, dipropylene glycol monoethyl ether acetate, 3-methoxybutanol acetate, tetrahydrofurfuryl acetate, and cyclohexanol acetate.

3. The solvent or solvent composition for organic transistor production according to one of claims 1 and 2,
wherein the organic semiconductor material comprises at least one compound selected from the group consisting of compounds (1) and compounds (2), the compounds (1) each represented by Formula (1): 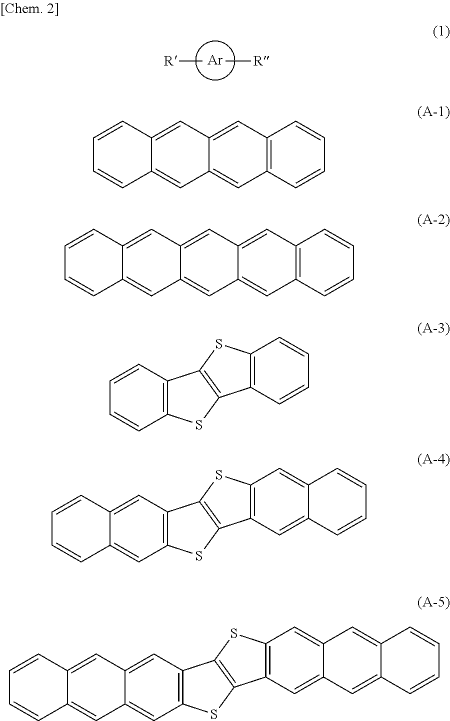
wherein Ar represents a group corresponding to a cyclic compound, except for removing two hydrogen atoms from the cyclic compound, where the cyclic compound is a compound represented by any one of Formulae (A-1) to (A-5); and R′ and R″ are each, identically or differently, selected from hydrogen, optionally substituted C₁-C₁₈ alkyl, optionally substituted phenyl, optionally substituted naphthyl, and optionally substituted thiophenyl, the compounds (2) each comprising at least one constitutional repeating unit represented by any one of Formulae (2-a) to (2-d): 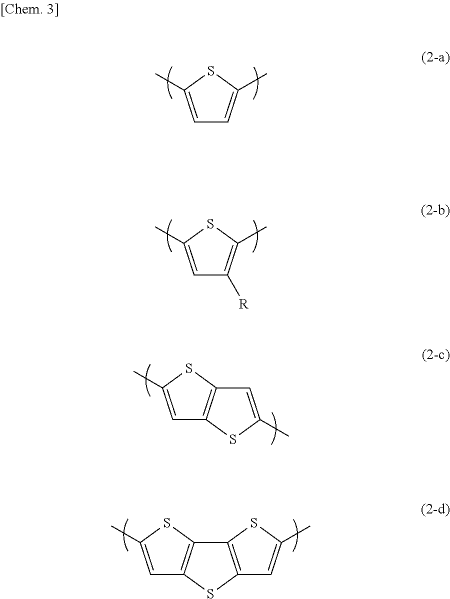
wherein R is selected from optionally substituted C₁-C₂₄ alkyl, optionally substituted phenyl, optionally substituted naphthyl, and optionally substituted thiophenyl.

4. A composition for organic transistor production, the composition comprising:
an organic semiconductor material; and
the solvent or solvent composition for organic transistor production according to one of claims 1 and 2.

5. The composition for organic transistor production according to claim 4,
wherein the organic semiconductor material comprises at least one compound selected from the group consisting of compounds (1) and compounds (2), the compounds (1) each represented by Formula (1): 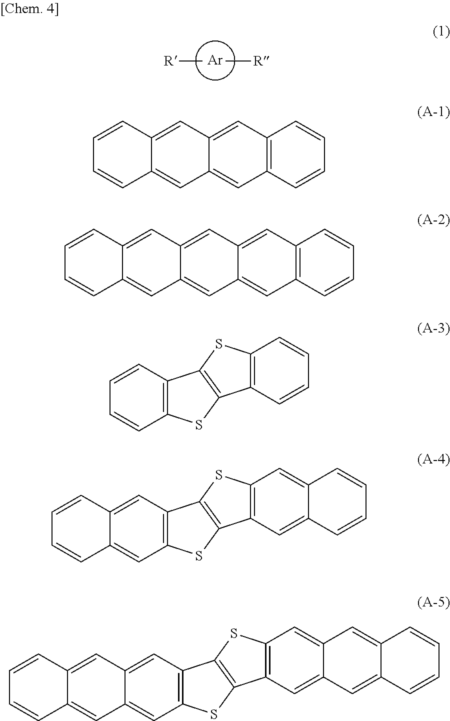
wherein Ar represents a group corresponding to a cyclic compound, except for removing two hydrogen atoms from the cyclic compound, where the cyclic compound is a compound represented by any one of Formulae (A-1) to (A-5); and R′ and R″ are each, identically or differently, selected from hydrogen, optionally substituted C₁-C₁₈ alkyl, optionally substituted phenyl, optionally substituted naphthyl, and optionally substituted thiophenyl, the compounds (2) comprising at least one repeating unit represented by any one of Formulae (2-a) to (2-d): 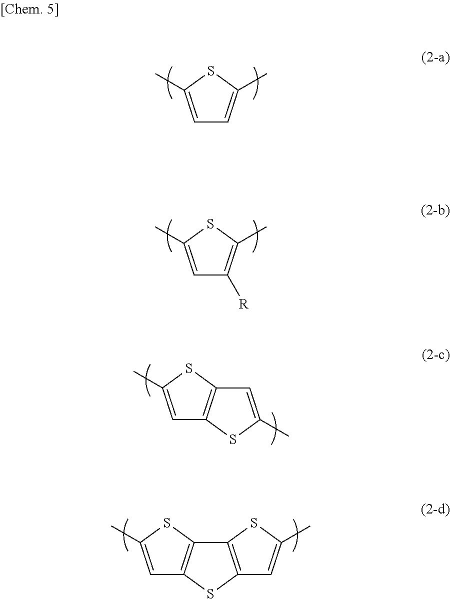
wherein R is selected from optionally substituted C₁-C₂₄ alkyl, optionally substituted phenyl, optionally substituted naphthyl, and optionally substituted thiophenyl.
